(19) United States
(12) Patent Application Publication
(10) Pub. No.: US 2024/0110925 A1
(43) Pub. Date: Apr. 4, 2024

(54) AUTOMATED REAL-TIME, ON-LINE N-GLYCOSYLATION MONITORING METHODS AND SYSTEMS THEREOF

(71) Applicant: Rutgers, The State University of New Jersey, New Brunswick, NJ (US)

(72) Inventors: Aron GYORGYPAL, New Brunswick, NJ (US); Shishir P.S. CHUNDAWAT, New Brunswick, NJ (US)

(21) Appl. No.: 18/375,243

(22) Filed: Sep. 29, 2023

Related U.S. Application Data
(60) Provisional application No. 63/412,064, filed on Sep. 30, 2022.

(51) Int. Cl. G01N 33/68; G01N 30/08; G01N 33/58

(52) U.S. Cl. CPC G01N 33/6854; G01N 30/08; G01N 33/582; G01N 2030/085; G01N 2440/38

(57) ABSTRACT

At least some embodiments disclosed here may be directed to design and implementation of a novel PAT tool for real-time (or near real-time) online N-glycosylation analysis through the use of sequential injection analysis (SIA) coupled with liquid chromatography; rapid and robust N-glycosylation monitoring during N-glycosylated protein (e.g., monoclonal antibody) bioprocessing enabled by instant, sensitive labeling chemistry; and systems thereof.

### CROSS REFERENCE TO RELATED APPLICATION

This application claims priority to U.S. Provisional Patent Application No. 63/412,064, filed Sep. 30, 2022, which is hereby incorporated by reference herein its entirety.

### STATEMENT REGARDING FEDERALLY SPONSORED RESEARCH OR DEVELOPMENT

This present disclosure was made with government support under grant number 1R01FD006588 awarded by the U.S. Food and Drug Administration (FDA). The government has certain rights in the present disclosure.

### FIELD OF DISCLOSURE

The present disclosure in various embodiments described here is generally directed to methods and systems for continuous biomanufacturing practices for, for example, manufacturing of biologics.

### BACKGROUND

The biopharmaceutical industry is moving towards incorporating continuous and other advanced biomanufacturing practices for manufacturing biologics and/or biological drugs. Typically, continuous bioprocesses may produce an increase in at least one of production flexibility, simplification scale-up processes, the quality of the end product, in productivity, or a reduction of the process footprint and/or production costs. There may be a need to shift biomanufacturing processes from a fed-batch process to a continuous process for purposes of manufacturing biologics.

Typically, N-glycosylation may be a quality attribute that can be tracked to control the function of antibodies and biologics. Unfortunately, available processes utilize offline quality control analysis for N-glycosylation of proteins and antibodies, which may be both time consuming, labor intensive, and expensive.

### SUMMARY

In accordance with the objectives disclosed herein, at least some embodiments of the present disclosure may facilitate shifting biomanufacturing processes from a fed-batch process to a continuous process for purposes of manufacturing biologics, by facilitating incorporation of technologies from multiple emerging fields such as, without limitation, bioprocess integration, automation, and/or digitization, as well as clear regulatory guidelines to facilitate this transition. The present disclosure provides in some objects, methods and systems defined by at least some embodiments of the present disclosure and in connection with examples provided here. In some other embodiments, some other features and/or advantages of the present disclosure will be apparent from the description.

As described here, in at least some embodiments, the present disclosure features methods and systems for manufacturing biologics, where the manufacturing practice may be continuous, automated, and under real-time for monitoring N-glycosylation.

### BRIEF DESCRIPTION OF FIGURES

FIGS. 1A-1B illustrate representative schemes of an embodiment described here, where a rapid, continuous, automated, and real-time biomanufacturing process is used to monitor and control the glycosylation process for further analyses.

FIG. 2 presents a representative flowchart of an embodiment described here.

FIG. 3A presents a representative flow scheme of the system described here, where the flow scheme comprises a portion of the system. The bioreactor (far left) and N-Glycanyzer are represented in the flow scheme. FIG. 3B illustrates an exemplary flow scheme of the valves (V1, V2) and syringe pumps (SP1, SP2). PB=Protein A phosphate buffer; Eluent=Protein A eluent; ProA=Protein A column; UV=UV spectrometer for titer and concentration analysis; Gly-X=Denaturant; IPC=detectable label; PNGase F WS=PNGase F:Working Solution, deglycosylation enzyme; ACN=Acetonitrile, polar aprotic solvent for trap column (HILIC); HEPES=4-(2-hydroxyethyl)-1-piperazineethanesulfonic acid, zwitterionic sulfonic acid buffering agent.

FIG. 4A shows a graphical scheme of an external valve and valve switching from a valve position of 1 to 6 to a valve position of 1 to 2. FIG. 4B illustrates a representative external valve with an image of Column integration, where the PEEK tubing (white arrow) is an outlet of N-GLYcanyzer (port 5) and waste from N-GLYcanyzer (port 6).

FIGS. 5A-5B show the separation of glycans and glycan analysis by fluorescent (FLR) analysis at excitation (ex.) peak at 285 nm and emission (em.) peak at 345 nm. Injection volume peak heights correlate to injection volume amount, i.e., 16 μL at ˜12 mins is the tallest peak (FLR ˜22.5); 8 μL at ˜ 12 mins (FLR ˜15.0); 4 μL at ˜12 mins (FLR ˜7.5); 2 μL at ˜12 mins (FLR ˜2.75); 1 μL at ˜12 mins (FLR ˜2.5). Inset graph: lines appear from top to bottom: G0F; G1F; G1F′. G0F is represented at retention time (RT) 12 mins; G1F at RT 14.5 mins, and G1F′ at RT 15.25 mins.

FIGS. 6A-6B illustrate the glycans eluted off of an analytical column from the trap column at variable wash volumes. FIG. 6B shows an enlarged portion from about Time 10 minutes to about Time 20 minutes of FIG. 6A. FIG. 6C presents a table showing integrated glycan peak abundances. A bias is demonstrated with the 1000 μL wash sample.

FIG. 7 compares a sample run with (50 mM HEPES, pH 8.0) or without (Formic acid/HEPES, pH 7.4) buffer exchange.

### DETAILED DESCRIPTION

Detailed embodiments of the present disclosure are disclosed herein; however, it is to be understood that the disclosed embodiments are merely illustrative of the present disclosure that may be embodied in various forms. In addition, each of the examples given in connection with the various embodiments of the present disclosure is intended to be illustrative, and not restrictive.

At least some embodiments of the present disclosure utilize at least one principle of an adaptation of a sensitive fluorescent and mass spectrometry detection with a labeling reagent composed of an activated form of procaine called Instant PC (IPC) (Agilent Technologies, Inc., Santa Clara CA) may be used in an online automated workflow for monitoring glycosylation (e.g., N-glycosylation) during, for example, monoclonal antibody bioprocessing.

One key challenge depends on real-time monitoring and control of the process, such as process analytical technology (PAT), which may facilitate important selection paths during bioprocessing dictated by monitoring critical quality attributes (CQAs). These CQAs may impact the efficacy and safety of the drug substance, and by monitoring key CQAs of the drug product and its intermediates during the entire manufacturing cycle, PAT ensures consistent quality of the final product. The methods and systems described here provide for improved product quality and optimization of the batch/fed-batch processes yielding high productivity (e.g., >10 g/L mAb titers) and further shifting into continuous manufacturing processes.

Illustrative Methods in Accordance with at Least Some Embodiments

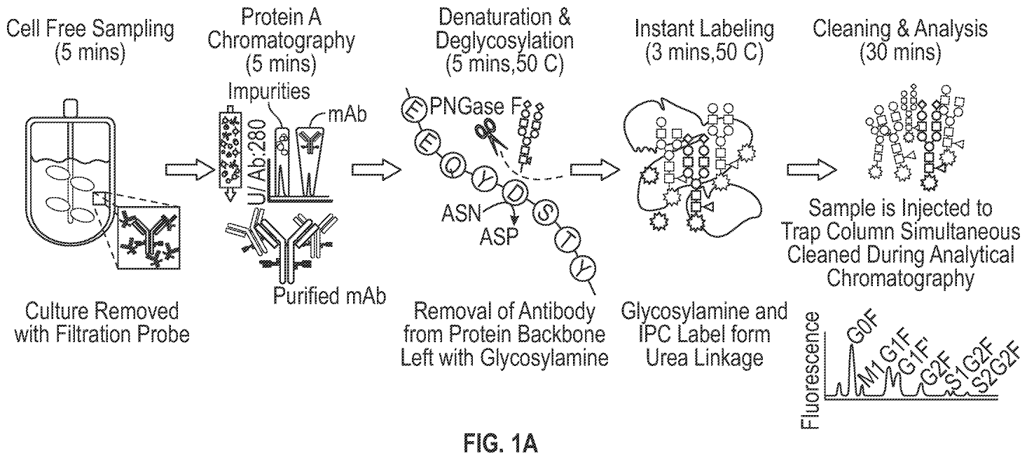

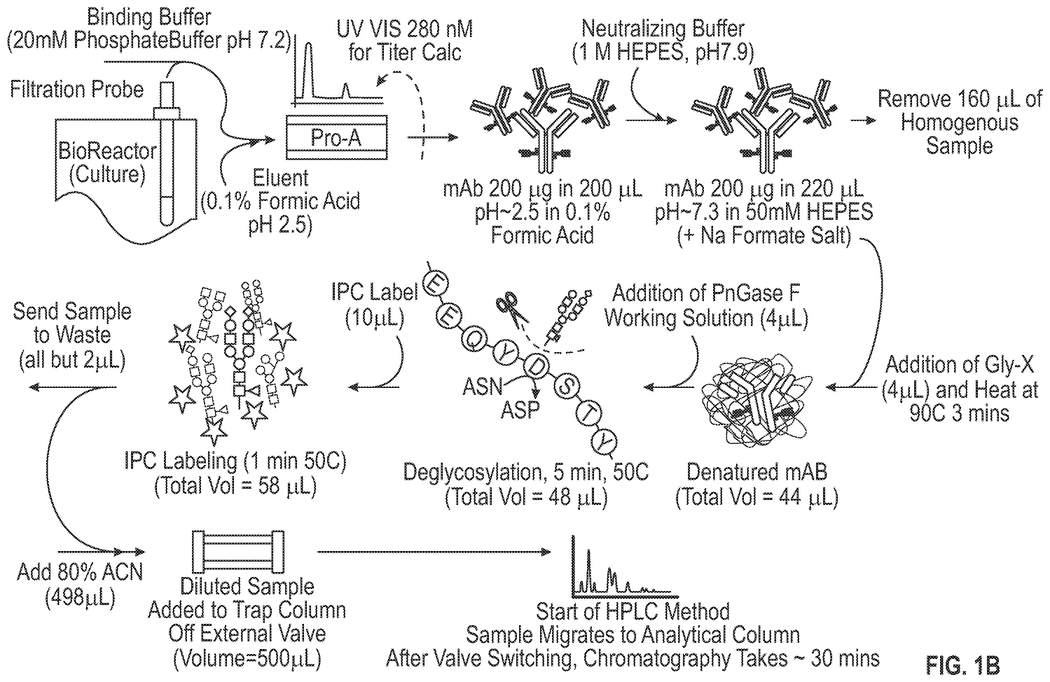

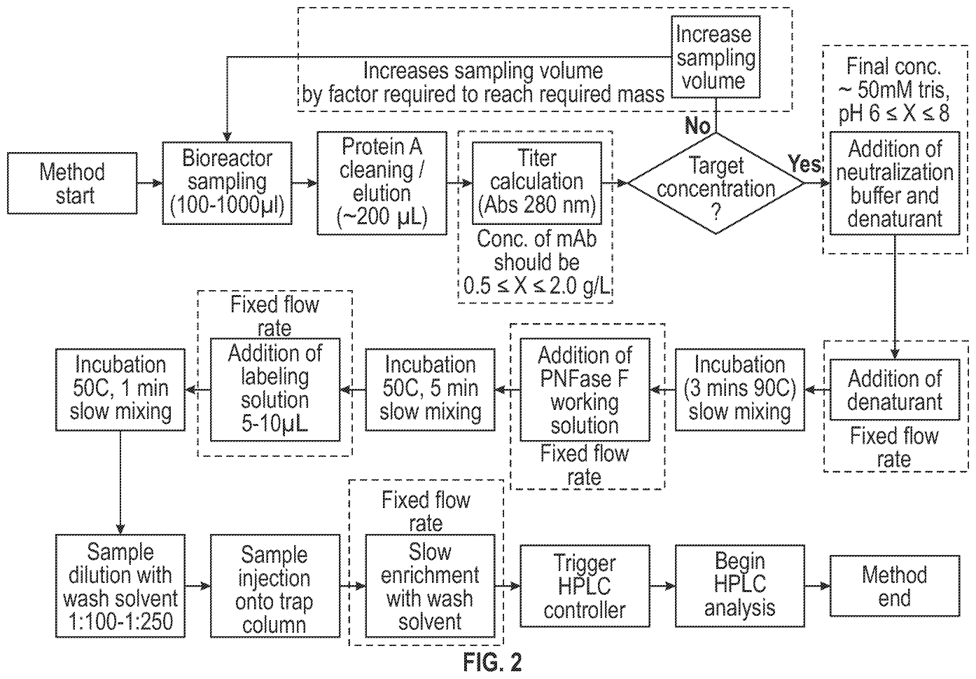

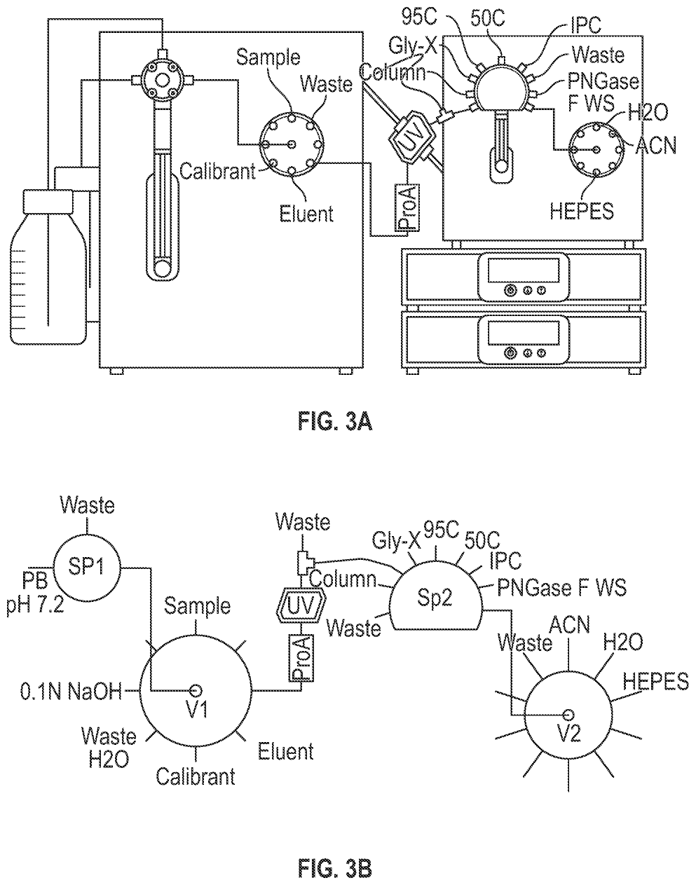

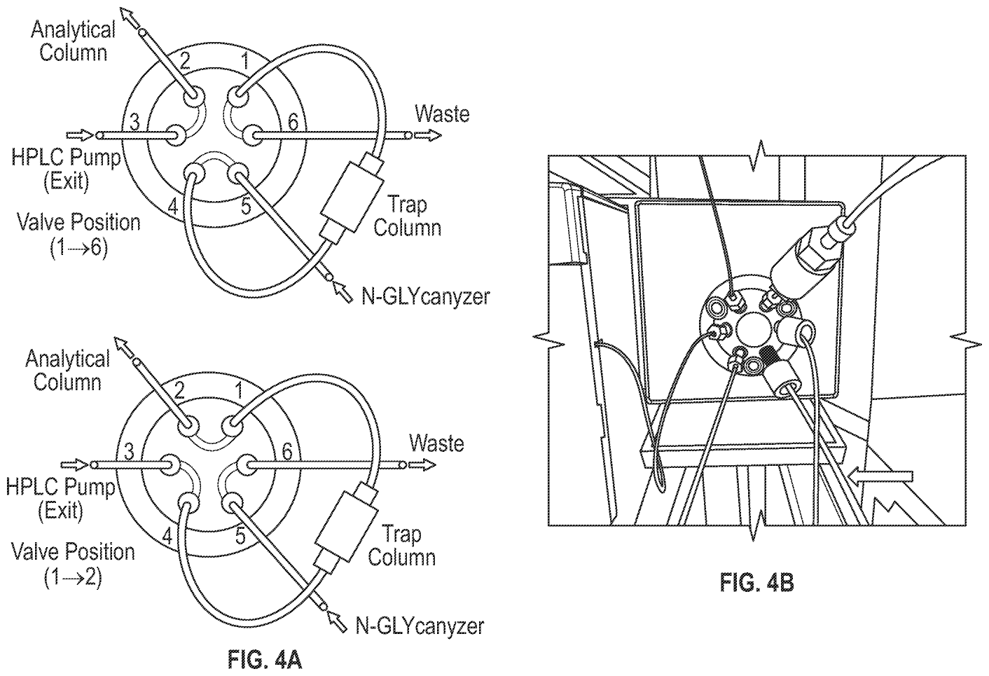

Provided here is a sequential injection, automated, online method and system for real-time analysis of glycoproteins, such as but not limited to, N-linked, O-linked, P-linked, S-linked, or C-linked glycan proteins. Embodiments described here may be exemplified with a particular glycoprotein, for example, N-linked glycan proteins, but may also be useful for other glycoproteins. In some embodiments, a method of monitoring and detecting glycosylation (e.g., N-glycosylation, O-glycosylation, P-glycosylation, S-glycosylation, C-glycosylation) of biologics provided here comprises: (a) obtaining glycoproteins or a plurality of glycoproteins (e.g., N-linked, O-linked, P-linked, S-linked, or C-linked); (b) purifying the glycoproteins (affinity chromatography, e.g., Protein A (ProA) column, ion exchange chromatography, hydrophobic interaction chromatography) (using e.g., ProSlAmpler system (FIAlab Instruments; Seattle, WA); SegFlow® Automated Sampling System (Flownamics; Madison, WI); MAST Cell Removal System; MAST®; Bend, OR) (c) measuring parameters (e.g., titer, concentration) of the glycoproteins (by e.g., UV spectroscopy flow cell); (optionally repeating steps (a)-(c)); (d) removing glycans from the glycoproteins (e.g., denaturing; deglycosylating); (e) labeling the glycans with a detectable label to form labeled glycans; (f) diluting the labeled glycans to a predetermined concentration to form diluted labeled glycans; (g) enriching the diluted labeled glycans on a trap column (e.g., HILIC column) to form enriched labeled glycans; and (h) detecting the enriched labeled glycans (e.g., fluorescence-based detection using, for example, HPLC: separating, detecting, identifying the enriched labeled glycans). See, e.g., FIGS. 1A-1B; FIG. 2; FIGS. 3A-3B; and FIGS. 4A-4B.

The glycoproteins or plurality of glycoproteins described here may be obtained from a bioreactor containing cells. Glycoprotein samples can also be obtained from cell broth secreted from cells, or alternatively, obtained from cell lysate if relevant to the manufacturing process. Some embodiments of the disclosure provide for a bioreactor, where cells are cultured to produce a plurality of glycoproteins (e.g., N-linked, O-linked, P-linked, S-linked, or C-linked glycoproteins), such as but not limited to Fc domain-containing proteins, including immunoglobulins, IgG, IgA, IgD, IgM, and IgE. The cells may include Chinese hamster ovary (CHO) cells which may be commonly used as an expression system for producing glycosylated recombinant proteins, e.g., N-glycosylated monoclonal antibodies, for manufacture of biotherapeutics. Instead of a batch (i.e., nutrient feeding only at the beginning of the cultivation process and none later; closed system) or a fed-batch (i.e., feeding with nutrients and supplements during culturing for high cell densities; partly open system) process, a continuous (i.e., constant cell density by steady state of incoming feed rate of growth-limiting substance rate of removal of harvest) culturing process as described here enables maximum productivity, reduction of cleaning, sterilization, and handling, and provides a steady state for metabolic studies. Exemplary and non-limiting disadvantages of the batch process includes a product comprising a mix of nutrients, reagents, cell debris, and toxins, and a short production time. Additional disadvantages for the fed-batch processes include, but are not limited to, an accumulation of inhibitory agents and toxin, potential contamination from the point of ingress, and production of a high cell density and product yields, which are problematic downstream by creating obstructions during the entire process. The continuous process as used in the methods and systems described here allow for continuous monitoring of the conditions of the bioreactor, thereby allowing adjustments and quality control during the manufacturing process and not at the conclusion of the manufacturing of the final product.

A cell-free sample (e.g., 100 μl-1000 μl) from, for example, a bioreactor may be obtained (for example, removed with a filtration probe) then purified and cleaned. The sample can also be obtained from cell broth secreted from cells, or alternatively, obtained from cell lysate if relevant to the manufacturing process. Purification of the glycoproteins (e.g., N-linked, O-linked, P-linked, S-linked, or C-linked glycoproteins) may occur by affinity chromatography (e.g., ProA, ion exchange, hydrophobic interaction chromatography) (using e.g., ProSlAmpler system (FIAlab Instruments; Seattle, WA); SegFlow® Automated Sampling System (Flownamics; Madison, WI); MAST Cell Removal System; MAST®; Bend, OR). For example, a Protein A column may be used to trap, clean, and enrich the glycosylated protein, e.g., monoclonal antibody (using, for example, the ProSIA system) under conditions to separate the impurities from the glycosylated proteins (e.g., monoclonal antibodies). The affinity column may be washed (e.g., sodium phosphate buffer) and eluted (e.g., formic acid). Elution of the N-glycosylated protein from the affinity column may utilize 100 μl or greater (e.g., 200, 300, 400, 500, 600, 700, 800, 900, 1000, 1100); 1000 μl or less (e.g., 950, 850, 750, 650, 550, 450, 350, 250, 150, 50); or 100 μl-1000 μl (e.g., 125 μl-975 μl; 175 μl-925 μl; 225 μl-925 μl; 275 μl-875 μl; 325 μl-825 μl; 375 μl-775 μl; 425 μl-725 μl; 475 μl-675 μl; 525 μl-625 μl). For example, the elution of glycosylated monoclonal antibodies from, for example, a Protein A affinity column may occur using about 200 μl. The titer of the eluant containing N-glycosylated proteins, such as the monoclonal antibodies, from the affinity column (e.g., ProA; ion exchange; hydrophobic interaction chromatography) may be determined by measuring the absorbance at 280 nm with a UV spectrometer. The concentration of the monoclonal antibodies should be about 0.5 g/L to about 2 g/L (i.e., 0.5 g/L≤X≤2 g/L). Titer and monoclonal antibody concentration may be determined by, for example, UV spectroscopy flow cell. The flow path of this embodiment allows for about 1 g/L of the N-glycosylated protein after the column elution step in the capillary. If the target concentration of the N-glycosylated protein has not been attained, the bioreactor sample volume (or cell broth sample volume or cell lysate volume) may be increased by a factor required to reach the required mass and the steps of purifying, titer calculation, and calculation of protein concentration repeated until the desired concentration is reached before proceeding.

After Protein A purification and the desired titer and protein concentration are achieved, the sample may be neutralized, before or during or after the denaturation step to a slightly alkaline pH, e.g., ˜pH 6-˜pH 8 (pH 7.9-pH 8), using HEPES (1 M HEPES, pH 7.9-pH 8). Also, raising the salt concentration to about 50 mM HEPES (˜pH 7.3) after Protein A elution using 0.1% formic acid allows the pH to be restored to this slightly alkaline pH necessary to mitigate glycosylamine hydrolysis. A final concentration of about 50 mM Tris and pH 6≤X≤pH 8 is, at least in some embodiments, desirable. It may also be desirable to reduce the salt concentration to as low as possible without affecting performance. The eluted N-glycosylated protein (200 μg/200 μL; pH ˜2.5 in 0.1% formic acid) may be neutralized (e.g., 1 M HEPES, pH 7.9) to result in N-glycosylated protein (e.g., 200 μg in 200 μL, pH ˜7.3 in 50 mM HEPES (plus sodium formate salt). A portion of the homogenous sample may be removed (160 μL). The remainder may also be denatured with the addition of denaturant (e.g., Gly-X; 4 μL) and heated at 90° C. for 3 minutes such that the denatured N-glycosylated protein (e.g., denatured monoclonal antibody; mAb) has a total volume of 44 μL. The denatured N-glycosylated protein may be deglycosylated under commonly known and understood conditions sufficient to remove the glycans from the N-glycosylated proteins, for example by enzymatic means such as PNGase F in working solution (4μL). For example, deglycosylation may occur at 50° C. for 5 minutes (total volume 48 μL). The activity of the enzyme (e.g., PNGase F) may be affected by temperature, pH, and salt concentration, which may be modified accordingly. The removal of protein, such as antibody, from the protein backbone to result in glycosylamine may occur by denaturing (e.g., using a surfactant at 90° C.) and deglycosylating (e.g., 5 mins, 50° C.) before labeling by the addition of, for example, a detectable label such as but not limited to, a fluorophore.

A sensitive, rapid labeling system for detection of the plurality of glycans removed from the plurality of N-glycosylated proteins (that are, e.g., denatured and deglycosylated monoclonal antibodies) may be useful, where the glycosylamines and label form a urea linkage under instant labeling conditions (e.g., 3 mins, 50° C.). The sensitive labeling system comprises, in some at least some embodiments, a detectable label (e.g., 2-(diethylamino)ethyl 4-((((2,5-dioxopyrrolidin-1-yl)oxy)carbonyl)amino)benzoate; 10 μL) for rapid labeling of the released and/or removed N-glycans. The labeling may occur at 50° C. for 1 minute and the concentration of detectable label (in a total volume of 58 μL) is important to prevent any bias and/or disruption in the downstream analysis. The detectable label produces a signal for glycans that make up as little as 0.1% of the profile and with a high signal-to-noise ratio. For example, signals having less than 1 luminescence unit (LU) are detectable. A portion (e.g., 2 μL) of the labeled glycans may be diluted with the addition of 80% acetonitrile (ACN) (e.g., 498 μL) to a pre-determined concentration (e.g., 1-40 μg; 2 μg-39 μg; 3 μg-38 μg; 4 μg-37 μg; 5-36 μg; 6 μg-35 μg; 7 μg-34 μg; 8 μg-33 μg; 9-32 μg; 10 μg-31 μg; 11 μg-30 μg; 12 μg-29 μg; 13 μg-28 μg; 14 μg-27 μg; 15 μg-26 μg; 16 μg-25 μg; 17 μg-24 μg; 18 μg-23 μg; 19 μg-22 μg; 20 μg-21 μg; 1 μg or greater; 40 or less) to form diluted labeled glycans. The remainder of the sample may be sent to waste.

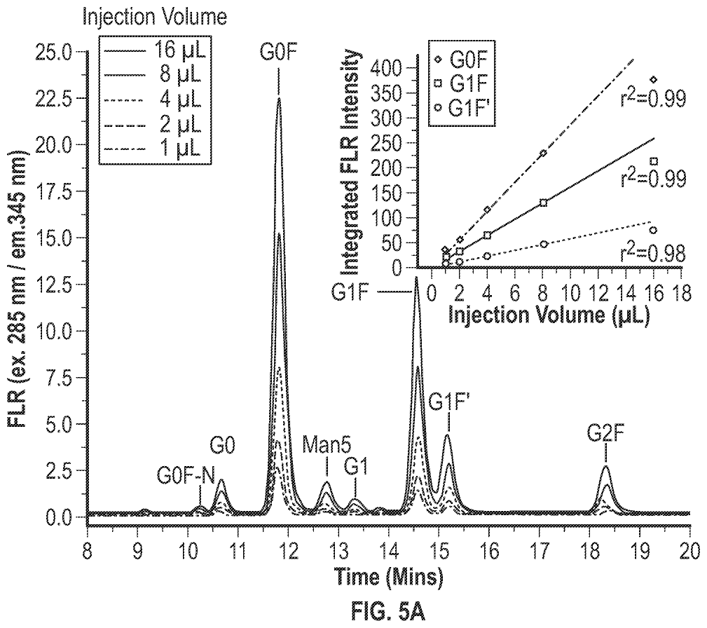

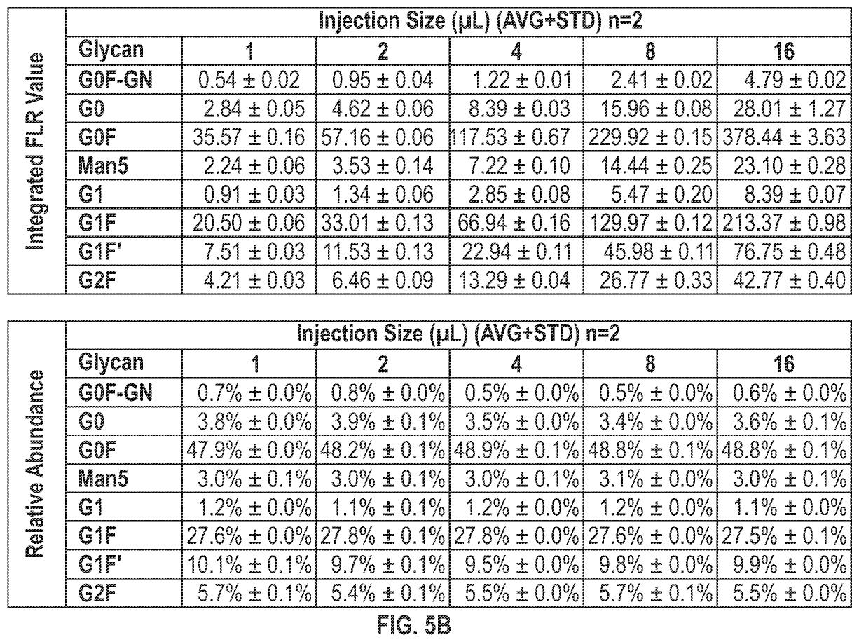

The diluted labeled N-glycans sample (e.g., 500 μL) is added to a trap column (e.g., hydrophilic interaction liquid chromatography (HILIC) column) off of an external valve. The trap column is, in some embodiments, fluidly connected to a loop allowing for sample enrichment without the loss of the analyte, while also removing excess fluorophores from the detectable label and any proteins that would interfere with the analytical workflow. Enrichment is not performed within a loop, but in the trap column, which may be connected to a loop. It was initially believed that a trap column would not necessarily work within this type of workflow but was successfully developed as described in various embodiments here after consideration of the flow rate and wash solution that were made compatible with both the trap column and analytical column disclosed here. The system of some embodiments described here may enable a bi-directional flow allowing for certain connected modules to be re-used as needed. Cleaning of the trap column may occur by injecting and washing different volumes of detectably labeled N-glycan samples within a 500 μL, matrix containing 80% acetonitrile (CAN) and 20% water. FIGS. 5A-5B demonstrate in some embodiments that there is no significant variation in residence time on the trap column and that the injection volume of prepared detectably labeled N-glycan sample does not cause a bias towards glycan relative abundances at lower injection volumes onto the trap column. The inset graph of FIG. 5A shows the injection volumes for the three major glycoforms from trastuzumab, a humanized monoclonal anti-human epidermal growth factor receptor 2 protein antibody. Information on integrated fluorescent values as well as relative abundances may also be observed.

The N-GLYcanyzer general process described in various embodiments here may include N-glycosylated protein capture (e.g., mAb capture), removal of glycans (e.g., denaturation, deglycosylation), glycan labeling (e.g., fluorescent), and glycan enrichment for direct injection and analysis on an integrated high performance liquid chromatography (HPLC) system.

### Illustrative System in Accordance with at Least Some Embodiments

In another embodiment, a system is provided here, where the system may comprise: (a) an affinity column (e.g., ProA column, ion exchange column, hydrophobic interaction chromatography) for purifying a plurality of glycoproteins (e.g., N-linked, O-linked, P-linked, S-linked, or C-linked glycoproteins); (b) a receptacle, where, for, for example, N-linked glycoproteins, the N-GLYcanyzer process occurs (e.g., N-glycosylated protein capture, denaturation of N-glycosylated protein, deglycosylation of denatured N-glycosylated protein, glycan labeling, and glycan enrichment); and (c) a valve system. The valve system described here may comprise: a valve and a trap column. See, e.g., FIGS. 4A-4B. The valve comprises, in some embodiments: at least one inlet from the receptacle (e.g., at a port at position 5 to 4 of the valve system), where the at least one inlet is fluidly connected to a trap column (at a port at position 1 to 6 of the valve system); and at least one outlet fluidly connected to an analytical column (e.g., Protein A resin column; at a port at position 1 to 2 of the valve system). The trap column (e.g., hydrophilic interaction liquid chromatography (HILIC)) may be fluidly connected to the at least one outlet. The affinity column (e.g., Protein A; ion exchange; hydrophobic interaction chromatography) of the system may be fluidly connected to the receptacle, and the receptacle fluidly connected to the valve system. The valve described here may be configured to redirect fluid flow between the at least one inlet and the at least one outlet. See, e.g., FIGS. 4A-4B. The valve may in some embodiments described here be fluidly connected to the receptacle, trap column, and the analytical column. The valve, in at least some embodiments, changes directions internally, which thereby changes the flow path between the N-GLYcanyzer system/receptacle and the analytical column. For example, the valve comprises at least one outlet that is fluidly connected to an analytical column, such as a Protein A resin column and subsequent analyzer.

A sample containing a plurality of glycoproteins (e.g., N-linked, O-linked, P-linked, S-linked, or C-linked glycoproteins), such as N-linked monoclonal antibodies, for use in the system, may be obtained from a bioreactor configured to comprise a cell culture under continuous processing. The sample can also be obtained from cell broth secreted from cells, or alternatively, obtained from cell lysate if relevant to the manufacturing process.

### Definitions

All terms used herein are intended to have their ordinary meaning in the art unless otherwise provided. All concentrations are in terms of percentage by weight of the specified component relative to the entire weight of the topical composition, unless otherwise defined.

As used herein, “a” or “an” shall mean one or more. As used herein when used in conjunction with the word “comprising,” the words “a” or “an” mean one or more than one. As used herein “another” means at least a second or more.

As used herein, all ranges of numeric values include the endpoints and all possible values disclosed between the disclosed values. The exact values of all half-integral numeric values are also contemplated as specifically disclosed and as limits for all subsets of the disclosed range. For example, a range of from 0.1% to 3% specifically discloses a percentage of 0.1%, 1%, 1.5%, 2.0%, 2.5%, and 3%. Additionally, a range of 0.1 to 3% includes subsets of the original range including from 0.5% to 2.5%, from 1% to 3%, from 0.1% to 2.5%, etc. It will be understood that the sum of all weight % of individual components will not exceed 100%.

Instances of “comprise” or “comprising” alternatively include “consist essentially” by which is meant that the components or ingredients include only the listed components and, for example, the normal impurities present in materials and with any other additives present at levels which do not affect the operation of embodiments of the present disclosure, for instance at levels less than 5% by weight or less than 1% or even 0.5% by weight.

The term “sensitivity” as used herein to describe the detection of labeled glycans may mean the detection of glycans at less than 1% of the glycan profile, for example as little as 0.1%. The detection method described here can, in some embodiments, detect the labeled glycans at a level that is 1 fold or greater (e.g., 2-fold; 3-fold; 4-fold; 5-fold; 10-fold; 100-fold; 1000-fold); 10,000-fold or less (e.g., 8000-fold; 6000-fold; 4000-fold; 2000-fold; 800-fold; 600-fold; 400-fold; 200-fold; 80-fold; 60-fold; 40-fold; 20-fold) than standard methods (for example using 2-AB).

### EXAMPLES

The following examples illustrate specific aspects of the instant description. The examples should not be construed as limiting, as the example merely provides specific understanding and practice of the embodiments and its various aspects.

### Example 1: Exemplary Flow Scheme

A sample from a bioreactor comprising cultured cells, such as CHO cells, was obtained and filtered through a 0.22 um filter. The sample was applied onto a ProA affinity column, cleaned using Phosphate buffer (e.g., pH 7.2), and then eluted with 0.1% Formic Acid (using a syringe pump 1 [SP1]). The sample titer was checked using a UV Abs280 spectrometer, and the eluted sample was flowed back through syringe pump 2 [SP2] and into valve 2 (V2) where HEPES was added to adjust the pH to ˜pH 8. Afterwards a denaturant was added, and the denaturing sample was incubated in a 95° C. heat chamber for denaturation. The denatured sample was removed, cooled, and a deglycosylation enzyme, PNGase F solution (1:1 PnGase F: Working solution), was added. Deglycosylation occurred in a 50° C. chamber and then removed. A detectable label, such as a fluorescent label, was added and incubated at 50° C. The sample was then homogenized, sent to waste, partly collected (˜5 μL or so), and diluted with 80% acetonitrile (CAN) (homogenized again) and sent to the trap column, then cleaned with 250 μL of 80% ACN. The trap column eluent was then sent to the HPLC side and chromatography was started. See, e.g., FIGS. 1A-1B; FIG. 2; FIGS. 3A-3B; and FIGS. 4A-4B.

FIGS. 5A-5B show the separation of glycans and glycan analysis of the sample injected into the analytical column, such as the HPLC column, by fluorescent (FLR) analysis at excitation (ex.) peak at 285 nm and emission (em.) peak at 345 nm.

### Example 2: HILIC Guard Column Specifications

Part Number: 821725-906|Particle Size: 2.7 um| Pore Size: 120 Å Inner Diameter: 2.1 mm Length: 5 mm

This design is modified from that described in Benet & Austin (Anal Biochem 414(1):166-168, 2011). The Benet & Austin system used the HPLC pump to clean off a trap column using a Waters BEH column that was connected through a trap column that ran in line with an analytical column. Their design required a gradient change to allow for their sample to fall off the column. For complete on-line integration, their requires to be 2 flow streams to allow for a sample handoff. However, their work does not explain the requirement for sample cleaning, nor do they quantify sample losses using their methodology. Unreactive 2-AB will competitively bind to their trap column and will not disassociate easily. A major issue with using the Benet & Austin methodology in its current form includes issues caused with peak shifting, for example holding the gradient isocratically for 2-5 minutes causes co-elution of different glycoforms during the analytical gradient. This becomes exacerbated with isomers and/or glycans that elute at close retention times such as G0F and Mannose 5, as well as the 2 isomers of G1F (G1Fa, G1Fb) and isomers of G1 (G1a, G1b). Benet & Austin also failed to show robustness or any data associated with complex glycoforms or siaylated glycans.

The inventive system and methods described here allow for sample hand-off, autonomously between the external 6 port valve and the N-GLYcanyzer. The elution profile and sample handoff between the HPLC and N-GLYcanyzer issues were solved by the described system and methods. The gradient and flow rates were critical for allowing disassociation of unreactive detectable label from the trap column (e.g., HILIC column) while retaining virtually 100% glycan retention with no peak broadening, or peak retention issues once switched between port positions 1-6 to 1-2. See, e.g. FIG. 4A. Multiple washing flow rates were analyzed in the workflow to allow for the cleaning of unreactive detectable label from the trap column. Flow rates of 2 μL/s-20 μL/s, different washings, concentrations between 70-90% Acetonitrile (ACN) with a balance percent of water or 50 mM ammonium formate were analyzed. It was determined that N-glycans eluted off the trap column as a result of pressure change between 2 bar and ˜300 bar once the valve switches from a 1→*6 position to a 1→2 position. A useful flow rate was determined to be approximately 5 μL/s, which was sufficient for the glycans to bind to the trap column. Moreover, a useful wash volume that was shown to be sufficient to remove excess unreacted detectable label off of the trap column was 500 μL. Significantly lower volumes were determined to be insufficient to clean the column properly and significantly higher volumes would cause higher glycoforms to prematurely elute off of the trap column.

### Example 3: Trap Column Injection

The purpose of the injection off column was to measure the binding capacity of the trap column, i.e., its ability to hold analyte in a non-biased way. Increasing the injection volume resulted in an increase in signal, which in turn allowed for fewer glycan species to be identified. The injection volumes of FIGS. 5A-5B refer to the volumes prior to dilution to a volume of 250 μL with wash solvent and addition to the trap column. For example, a 16 μL injection volume was a 16 μL sample containing labeled glycans+234 wash solvent, and a 1 μL injection volume was a 1 μL sample containing labeled glycans+249 μL wash solvent. All of the injected samples were then washed with an additional 250 μL of washing solvent before analyte was added to and flowed onto the trap column. The steps prior to the trap column occurred in a receptacle.

It should be noted that a volume larger than a 16 μL sample injection volume caused bias to the column. Some glycoforms favored a longer retention time on the trap column during the wash and/or elution phase. For example, larger glycoforms had an increased affinity to the trap column, which causes bias in the sample. Accordingly, smaller injection volumes, such as but not limited to, 8 μL or less (e.g., 7, 6, 5, 4, 3, 2, 1, 0.5, 0.25); 1 μL or greater (e.g., 1.5, 2.5, 3.5, 4.5, 5.5, 6.5, 7.5, 8.5, 9.5); or 1 μL-10 μL, (e.g., 2 μL-9 μL; 3 μL-8 μL; 4-7 μL; 5-6 μL) pre-dilution should be injected onto the trap column to mitigate this observed bias.

For FIGS. 5A-5B, all of the samples were from the same sample preparation to mitigate any bias that may be introduced during the prior steps of the sample preparation (i.e., the N-glycosylated protein (e.g., mAb sample), denaturation, deglycosylation, labeling, etc.).

### Example 4: Trap Column Washing

The volume of wash required to remove excess fluorophore from the trap column prior to elution to the analytical column (e.g., Protein A resin column) was determined. A wash volume that was too small resulted in fluorescent dampening of critical glycans under some circumstances and did not allow early elution peaks (of the glycans) to be seen and identified. A wash volume that was too harsh and/or stringent caused adsorption issues onto the column and some glycoforms eluted off the trap column prior to switching over to the analytical column (e.g., Protein A resin column) once again causing bias.

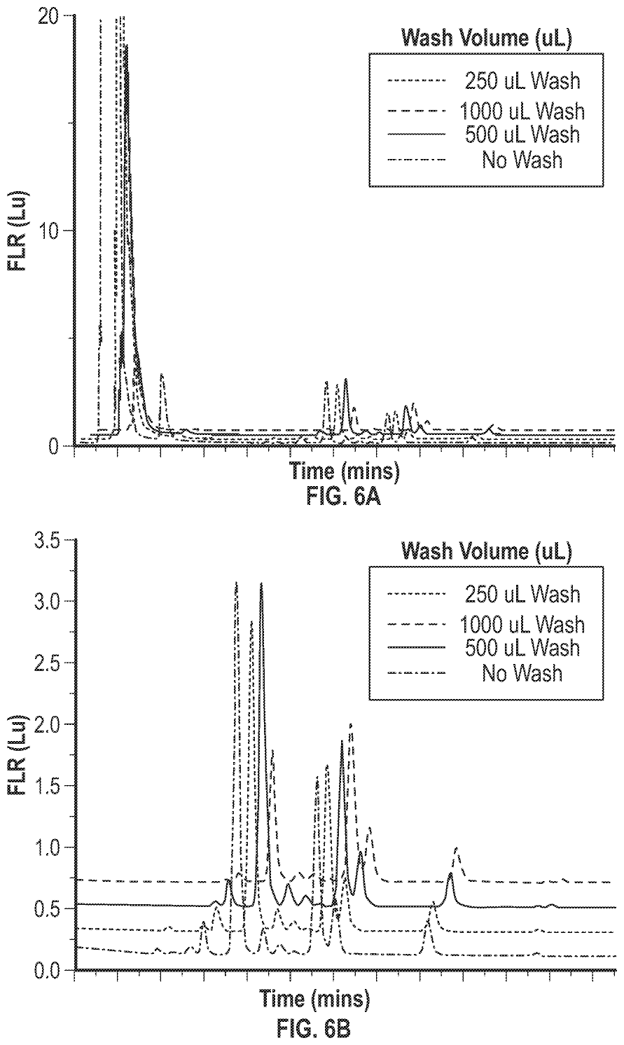

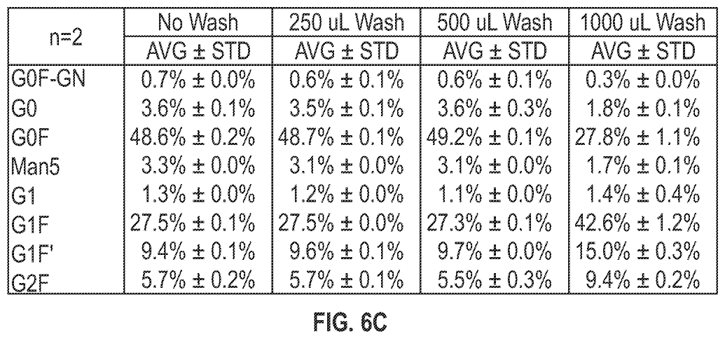

FIGS. 6A-6B demonstrate that a wash volume between 250 μL-500 μl, with a wash solvent resulted in an optimal ration and base line separation without a loss in the glycan ratios. FIGS. 6A-6B show graphs illustrating the glycans that eluted off of the analytical column (e.g., Protein A resin column) from the trap column at variable wash volumes. The washes occurred in the N-GLYcanyzer receptacle through a 6-port valve.

FIG. 6A presents a chromatograph of the elution of the free detectable label (e.g., IPC labeling dye) with wash volumes or none. The No Wash sample shows an excess of free detectable label with a very high fluorescence as measured by light unit (Lu). The graphical representation of free detectable label in a No Wash sample eluted at time 0.5 minutes and is the front-most graphical line. The 1000 μL, wash volume had the smallest peaks and was the back-most graphical line. The increase in wash volume resulted in a decrease amount of the free detectable label (e.g., IPC dye) from the analytical column. FIG. 6B shows the separation of glycans in an enlarged portion of the chromatogram of FIG. 6A. The No wash, 250 μL wash, and 500 μL wash samples had similar peak heights. The 1000 μL wash introduced a bias as demonstrated by its decreased peak height. The table of FIG. 6C similarly shows the bias with the 1000 μL wash.

### Example 5: Sample Preparation Analyses

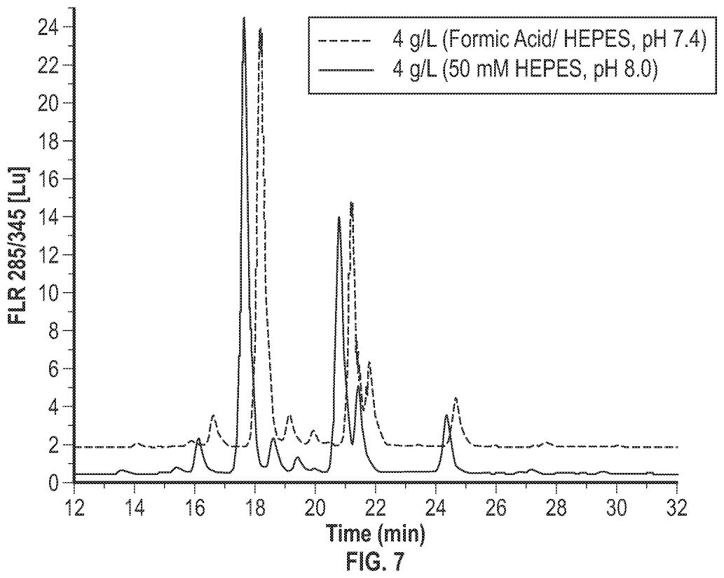

The N-GLYcanyzer system cannot run a buffer exchange. Accordingly, there were concerns that the online process described here may affect the samples. For example, there were concerns that the non-alkaline condition would cause issues with glycosylamine degradation after mAb protein deglycosylation. Therefore, the absence of a buffer exchange affecting workflow by potentially introducing bottlenecks or obstacles was investigated. Samples were run with or without a buffer exchange. A comparison of denaturation, deglycosylation efficiency, and label efficiency in the presence or absence of buffer exchange was performed. A sample of monoclonal antibody was buffer exchanged into a 50 mM HEPES, pH 8.0 solution before denaturation sample preparation. Another sample was purified by ProA affinity chromatography column and then neutralized using 1 M HEPES, pH 7.9 to bring the sample pH to ˜pH 7.4 (e.g., formic acid/HEPES, pH 7.4). FIG. 7 compares the fluorescence intensity (FLR) comparing samples with buffer exchange (lower graphical peak line; at 12 mins, ˜0.5 FLR) to without buffer exchange (upper graphical peak line; at 12 mins, ˜2 FLR). Since the FLR intensity between the two samples were similar, it was determined that the lack of a buffer exchange did not affect the workflow performance.

### Specific Embodiments

Non-limiting specific embodiments are described below, each of which is considered to be within the present disclosure.

1. A method, comprising:
(a) obtaining a plurality of glycoproteins (e.g., N-linked, O-linked, P-linked, S-linked, or C-linked glycoproteins) (e.g., Fc domain-containing antibodies: IgG, IgA, IgD, IgM, IgE) (from cultured cells);
(b) purifying the glycoproteins (e.g., N-linked, O-linked, P-linked, S-linked, or C-linked glycoproteins) (by affinity chromatography, e.g., Protein A column; ion exchange column; hydrophobic interaction chromatography) (e.g., using ProSIA system);
(c) measuring parameters (e.g., titer, concentration) of the glycoproteins (e.g., N-linked, 0-linked, P-linked, S-linked, or C-linked glycoproteins) (e.g., UV spectroscopy flow cell);
(optionally repeating steps (a)-(c));
(d) removing glycans from the glycoproteins (e.g., N-linked, O-linked, P-linked, S-linked, or C-linked glycoproteins) (e.g., denaturing; deglycosylating);
(e) labeling the glycans with a detectable label to form labeled glycans;
(f) diluting the labeled glycans to a pre-determined concentration to form diluted labeled glycans;
(g) enriching the diluted labeled glycans on a trap column (e.g., HILIC column) to form enriched labeled glycans; and
(h) detecting the enriched labeled glycans (e.g., fluorescence-based detection; HPLC: separating, detecting, identifying enriched labeled glycans).

2. The method of embodiment 1, wherein the plurality of glycoproteins (e.g., N-linked, O-linked, P-linked, S-linked, or C-linked glycoproteins) is from a bioreactor.

3. A system, comprising:
(a) an affinity column (e.g., Protein A) for purifying glycoproteins (e.g., a plurality of N-linked glycoproteins);
(b) a receptacle;
(c) a valve system,
   wherein the valve system, comprises:
      a valve, comprising:
         at least one inlet from the receptacle (at a port at position 5 to 4 of the valve system),
         wherein the at least one inlet is fluidly connected to a trap column (at a port at position 1 to 6 of the valve system); and
         at least one outlet fluidly connected to an analytical column (e.g., Protein A resin column; at a port at position 1 to 2 of the valve system); and
      a trap column (e.g., hydrophilic interaction liquid chromatography (HILIC)),
         wherein the trap column is fluidly connected to the at least one outlet;
   wherein the affinity column is fluidly connected to the receptacle;
   wherein the receptacle is fluidly connected to the valve system;
      wherein the valve is configured to redirect fluid flow between the at least one inlet and the at least one outlet.

4. The system of embodiment 3, wherein the plurality of N-linked glycoproteins is obtained from a bioreactor (configured to comprise a cell culture); a cell broth; or a cell lysate.

5. The system of embodiment 3, wherein the analytical column is a Protein A resin column.

As various changes can be made in the above-described subject matter without departing from the scope and spirit of the present disclosure, it is intended that all subject matter contained in the above description, or defined in the appended embodiments and/or claims, be interpreted as descriptive and illustrative of the present disclosure. Many modifications and variations of the present disclosure are possible in light of the above teachings. Accordingly, the present disclosure is intended to embrace all such alternatives, modifications and variances which fall within the scope of the appended embodiments and/or claims.

All documents cited or referenced herein and all documents cited or referenced in the herein cited documents, together with any manufacturer's instructions, descriptions, product specifications, and product sheets for any products mentioned herein or in any document incorporated by reference herein, are hereby incorporated by reference, and may be employed in the practice of the disclosure.

What is claimed is:

1. A method, comprising:
(a) obtaining a plurality of N-linked glycoproteins;
(b) purifying the N-linked glycoproteins;
(c) measuring parameters of the N-linked glycoproteins;
(optionally repeating steps (a)-(c));
(d) removing glycans from the N-linked glycoproteins;
(e) labeling the glycans with a detectable label to form labeled glycans;
(f) diluting the labeled glycans to a pre-determined concentration to form diluted labeled glycans;
(g) enriching the diluted labeled glycans on a trap column to form enriched labeled glycans; and
(h) detecting the enriched labeled glycans.

2. The method of claim 1, wherein the plurality of N-linked glycoproteins is from a bioreactor.

3. The method of claim 1, wherein the plurality of N-linked glycoproteins is from cultured cells.

4. The method of claim 1, wherein the plurality of N-linked glycoproteins comprises Fc domain-containing antibodies.

5. The method of claim 1, wherein the plurality of N-linked glycoproteins comprises Fc domain containing antibodies selected from: IgG, IgA, IgD, IgM, IgE, or any combinations thereof.

6. The method of claim 1, wherein purifying occurs by affinity chromatography.

7. The method of claim 6, wherein purifying occurs by Protein A column; ion exchange column; or hydrophobic interaction chromatography.

8. The method of claim 1, wherein the parameters comprise titer and concentration.

9. The method of claim 1, wherein measuring is by UV spectroscopy flow cell.

10. The method of claim 1, further comprising optionally repeating (a), (b), and (c) before (d) removing glycans.

11. The method of claim 1, wherein removing glycans occurs by denaturing, deglycosylating, or any combinations thereof.

12. The method of claim 1, wherein the trap column is a hydrophilic interaction liquid chromatography (RELIC) column.

13. The method of claim 1, wherein detecting occurs by fluorescence-based detection.

14. The method of claim 1, wherein detecting further comprises:
separating;
detecting; and
identifying the enriched labeled glycans.

15. A system, comprising:
(a) an affinity column for purifying a plurality of N-linked glycoproteins;
(b) a receptacle;
(c) a valve system,
wherein the valve system, comprises:
a valve, comprising:
at least one inlet from the receptacle,
wherein the at least one inlet is fluidly connected to a trap column; and
at least one outlet fluidly connected to an analytical column; and
a trap column,
wherein the trap column is fluidly connected to the at least one outlet;
wherein the affinity column is fluidly connected to the receptacle;
wherein the receptacle is fluidly connected to the valve system;
wherein the valve is configured to redirect fluid flow between the at least one inlet and the at least one outlet.

16. The system of claim 15, wherein the plurality of N-linked glycoproteins is obtained from a bioreactor (configured to comprise a cell culture); a cell broth; or a cell lysate.

17. The system of claim 15, wherein the analytical column is a Protein A resin column.

18. The system of claim 15, wherein the at least one inlet from the receptacle is at a port at position 5 to 4 of the valve system.

19. The system of claim 15, wherein the at least one inlet is fluidly connected to the trap column at a port at position 1 to 6 of the valve system.

20. The system of claim 15, wherein the at least one outlet is fluidly connected to the analytical column at a port at position 1 to 2 of the valve system.
